(19) United States
(12) Patent Application Publication
(10) Pub. No.: US 2026/0104360 A1
(43) Pub. Date: Apr. 16, 2026

(54) DETECTING HYDROGEN BY MEASURING AMPLIFIED HYDRIDE CRYSTALLOGRAPHIC CHANGES

(71) Applicants: Christian Adams, Yalaha, FL (US); Jody Wilson, Grand Island, FL (US)

(72) Inventors: Christian Adams, Yalaha, FL (US); Jody Wilson, Grand Island, FL (US)

(73) Assignee: Multicore Technologies, LLC, Orlando, FL (US)

(21) Appl. No.: 18/914,219

(22) Filed: Oct. 13, 2024

(51) Int. Cl. G01N 21/77; G01N 33/00

(52) U.S. Cl. CPC G01N 21/7703; G01N 33/005; G01N 2021/7779; G01N 2201/088

(57) ABSTRACT

A system and method is disclosed for providing a hydrogen sensor optimally comprising a multicore optical fiber strain sensor, interacting with a kinematic or mechanical amplification assembly, which is in turn connected to a responsive hydrogen storage material. In general, the sensor comprises any system or method whereby hydrogen concentration is derived from measuring the strain associated with crystallographic change and/or displacement associated with a hydrogen storage material as it takes up and gives off hydrogen.

### BACKGROUND OF THE INVENTION

### 1. Technical Field

Embodiments disclosed herein relate generally to sensors, sensing methods, and applications using the sensors and sensing methods including the detection of hydrogen; more particularly, to strain sensors with design options including optical fiber, specifically multicore fiber (MCF) or Fabry Perot fiber-optic interferometric sensors; or Mach-Zehnder interferometric sensors, or Michelson interferometric sensors, or conventional Wheatstone bridge electrical strain sensors, or even electromagnetic radiation-based displacement sensors that can potentially measure strain indirectly, including but not limited to millimeter wave or laser-based displacement detection mechanisms, or 3D scanning technologies. These sensors may, in turn, receive amplified inputs through kinematic or mechanical actuation from hydrogen-absorbing materials that undergo reversible crystallographic strain-inducing phase changes upon hydrogen exposure, thus yielding a hydrogen sensor when properly calibrated.

### 2. Description of Prior Art

Because hydrogen is an odorless, colorless, and tasteless gas, various groups including industry, academia, and government rely on hydrogen gas detectors to detect uncontrolled leaks or, in controlled environments, to determine otherwise unknown concentrations of hydrogen. Different legacy hydrogen sensor types include Pellistor (catalytic bead), electrochemical, semiconductor, and thermal conductivity sensors.

Pellistor sensors rely on the use of a catalyst, typically platinum, that causes reactive gases that contact the bead sensor to undergo an exothermic reaction. When this reaction occurs, heat is produced in proportion to the amount of flammable gas present and is not specific to hydrogen. Pellistor gas detectors are currently the most robust, reliable, and cost-effective detectors of their kind, making them a popular and reliable choice for hydrogen gas detection. Electrochemical sensors, on the other hand, use an electrolyte to generate a current proportional to the amount of gas detected. This method provides a more sensitive hydrogen gas detection mechanism than that of Pellistor sensors, but the technology is prone to damage or degradation when exposed to target gas levels exceeding their measurement range. Another technology, metal oxide semiconductor gas detectors, can detect hydrogen but this technology also responds to a wide range of other gases and vapors so are not advisable for hydrogen detection applications due to the likelihood of false alarms. Last and least, thermal conductivity sensor is another technology, but low sensitivity and low selectivity make this approach a poor choice for hydrogen detection.

Various fiber optic-based hydrogen sensors can be found in literature, with several enjoying protected intellectual property, and most employing palladium (Pd) as the target metal for absorbing hydrogen. When Pd is exposed to molecular hydrogen, it catalytically dissociates into atomic hydrogen on the Pd surface. Because hydrogen atoms diffuse easily through a Pd film, the metal is rapidly converted into palladium-hydride with the H held inside the Pd lattice by van-der-Waals forces. The Pd:H ratio depends on the partial pressure of hydrogen gas and involves a reversible crystallographic phase transition involving an increase or decrease in the metal's unit cell size. This phase transition provides the measurable stress detectable with fiber-optic-based measurement by different patented mechanism including the inventive disclosure.

Fiber optic-based hydrogen sensors are also becoming more common, FIG. 1 provides an overview of different types of fiber optic based-sensors. They all take advantage of a hydride material, typically palladium (Pd), and can in turn be sub-classified according to their working principle: (a) interferometric-based (Mach-Zehnder, Fabry-Perot); (b) intensity-based; or (c) fiber Bragg grating-based sensors [1].

European patent EP258434A1 describes a fiber optic hydrogen sensor that has been prepared in such a manner that exposure to specific pretreatment environment regimen results in a “pre-attenuation” of the fiber optic light signal. This pretreatment yields a reduction in the light intensity over time because of exposure to hydrogen, but no further attenuation takes place resulting from addition exposure to the reducing hydrogen environment; this is an intensity-based detection mechanism.

U.S. Pat. No. 9,322,969B2, “Hydrogen-sensing optical fiber hydrogen-passivated to prevent irreversible reactions with hydrogen and hydrogen-induced attenuation losses” is noted as a method to treat any optical-fiber based hydrogen sensor since it's well known that exposure of the silica fiber to hydrogen atmosphere can impact the index of refraction of the different parts of the fiber optic makeup, therefore imparting an ongoing drift as the hydrogen analyte changes the performance of the sensor because of this change in the index of refraction. This is because the internal reflection of the light signal in a fiber optic is dependent on index of refraction differences. By passivating the fiber in hydrogen prior to deployment this drift is mitigated.

Patent #US20040173004A1 discusses the application of different layers of zeolite on the surface of the active hydrogen-detecting portion of the optical fiber, in this case catalytic and hydrogen absorbing palladium. Zeolite material is porous at the molecular and even atomic scale, thereby mitigating the ingress of potential poisons such as water vapor, ammonia, methane, carbon monoxide, and/or carbon disulfide that may be present at the point of measurement. This improves the performance and reliability of the sensor by eliminating substances that can interfere with the hydrogen absorption process; this being the hydrogen uptake process in palladium that causes a change in Pd resistance as measured by a Wheatstone bridge circuit.

U.S. Pat. No. 8,547,553B2 describes a fiber optic sensor that employs “hydridable” materials (compounds that exhibit the ability to absorb/adsorb hydrogen into the unit cell structure), specifically a multilayered sensing film reactive to hydrogen and positioned about the fiber cladding, comprising a modulated structure comprising a plurality of hydridable material layers, and alternating with a plurality of non-hydridable material layers. This fiber optic technique utilizes fiber Bragg grating sensors to detect thermal differences and refractive index variations stemming from this periodic modulation of the grating structures that comprise overall a long-period fiber grating structure.

U.S. Pat. No. 8,842,944B2, “Optical fiber hydrogen sensor and method of measuring hydrogen concentration using the same,” describes an optical fiber hydrogen sensor and a method of measuring a hydrogen concentration that includes a light source, a polarization beam splitter connected to the light source to split light from the light source into two polarization beams, a high-birefringence optical fiber connected to the polarization beam splitter and coated with a hydrogen reactant, and an optical spectrum analyzer connected to the polarization beam splitter to measure an interference spectrum generated at the high-birefringence optical fiber. This interference spectrum results when a “high-birefringence optical fiber” is coated with an H2 sensitive material selected to strain the fiber upon hydrogen absorption. A second optical fiber is also required. This birefringence pattern requires subtracting the signal from the coated fiber optic and the reference fiber optic using a spectrum analyzer; a shift in this pattern can be mapped to hydrogen concentration.

Although there has been some progress in technology, features, and refinement over time, and some of the recent intellectual property filings provide ways to protect and/or add specificity to the hydrogen sensitive components of the sensors, none of the existing technologies or listed inventions provide a simple, low-cost, optical-fiber solution for detecting hydrogen. What is needed, therefore, is an inexpensive, robust, and compact method to leverage fiber optic technology to the field of hydrogen detection. Because multicore fiber (MCF) sensor technology provides an extremely sensitive platform for measuring various parameters including bend and temperature, this platform can measure hydrogen concentration reliably and inexpensively using the inventive description contained herein.

### SUMMARY OF THE INVENTION

An improved hydrogen concentration sensor comprising a strain sensor actuated by a responsive hydrogen-absorbing metallic hydride material. Strain sensors are not new, but utilizing strain sensors to measure crystallographic distortions that manifest in a sample of metal hydride responding to changes in hydrogen concentration is novel; these distortions reversibly manifest as a function of hydrogen concentration found in a gas flow that the hydride material is subjected to. Specifically, the invention leverages mechanical or kinematic amplification to translate these reversible crystallographic distortions onto a strain sensor of any kind, whether conventional, interferometer based, or fiber-optic based.

Flexure-based displacement amplification mechanisms (FDAMs) represent a valuable solution for achieving ultra-precision actuation while necessitating a balanced consideration of factors such as travel range, system complexity, and manufacturing costs. As such their application can be taken advantage of as part of the inventive device. FDAMs substantially augment the effective stroke of Precision Actuation Systems (PSAs), such as one possible and non-limiting design described herein, through their inherent amplification properties, such as that which is needed for amplifying subtle changes to the dimensions of a hydrogen absorbing material including hydrides. These amplification mechanisms are characterized by flexure hinges that offer notable advantages, including the absence of friction and zero backlash, thereby preserving high precision, and minimal assembly requirements, leading to reduced system costs [2].

### DESCRIPTION OF THE DRAWINGS (SEE DRAWINGS SECTION)

FIG. 1 provides an overview of different categories of optical fiber-based sensors that utilize hydride materials to measure hydrogen concentration.

FIG. 2 shows an optical circulator diagram, which is an arrangement of optical components that direct the optical signal from one port to another in one direction, in this case one input and two outputs.

FIG. 3 provides a notional diagram of typical multicore fiber (MCF) sensor arrangement inserted as part of a standard telecom single mode optical fiber.

FIG. 4 provides a diagram of one notional mechanical amplification layout consisting of a hydride material that transfers hydrogen concentration-related dimensional changes through an actuation mechanism to a strain sensor, in this case an MCF optical sensor.

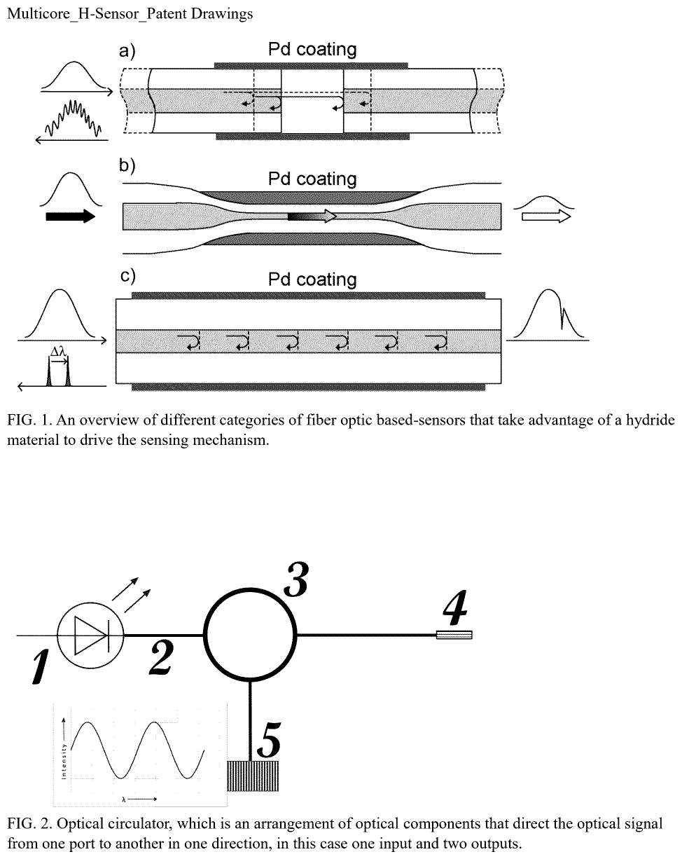

### DETAILED DESCRIPTION OF THE INVENTION

The multicore fiber (MCF) sensor is a recent addition to the family of sensors derived from fiber optic communication technology. As such, the MCF sensor benefits by being visually indistinguishable from conventional silica fiber optic cable and can take advantage of remote sensor placement along the length of or at the end of a common single mode optical fiber run. Referring first to FIG. 2 of the drawings, a diagram shows those familiar with the art of fiber optic communications a simple optical circulator assembly with a light source 1 launching light into the input fiber port 2, through the circulator 3 into the MCF sensor 4 which has a reflecting facet at the terminus, returning a modified signal back to the circulator and into the oscilloscope 5 and displaying the associated wave form. This wave form spans a short frequency range, usually including the 1550 nm range of the infra-red, therefore leveraging common light sources and detection equipment as this is among the most common wavelengths found in the telecommunications world. As mentioned in the Summary, the wave form modulation may have up to a 50 dB dynamic range depending on the design of the MCF and the oscilloscope output 5 is high contrast and easy to measure. The signal is strong enough to allow for a narrow bandpass filter to be employed allowing for the signal to be distilled into a single brightness level at a narrowed bandwidth and therefore measurable by a common photodetector. Given that light source 1 can be a LED, and detector 5 can be a photodiode, the sensing package is extremely innovative because of its simplicity and low cost of the parts involved, all while benefitting from the patent describing the MCF optic sensor, U.S. Pat. No. 9,810,557.

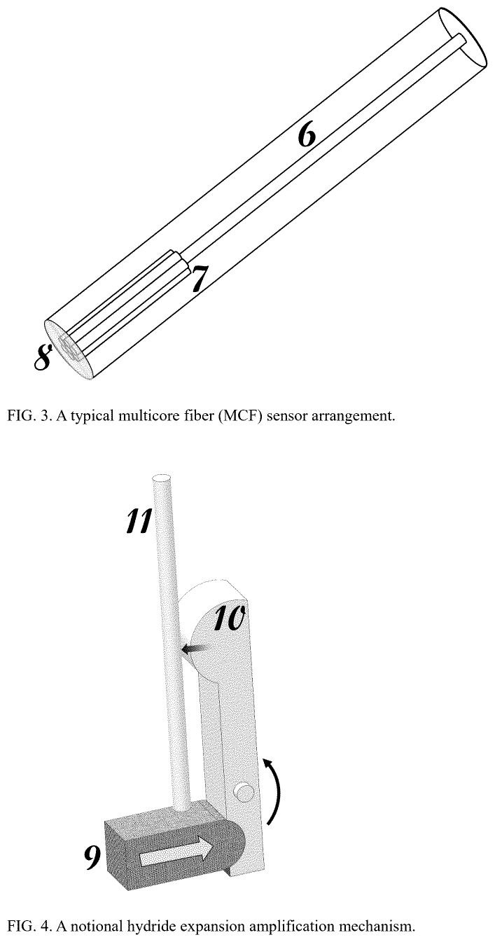

Changes in certain physical parameters of interest in an external environment where the MCF device, referred to in FIG. 3, connected via the aforementioned circulator hardware through single mode fiber (SMF) 6 into a fusion-spliced fiber section of 7-core MCF sensor 7, whereby the environment immediately around the sensor affects the sinusoidal multimode interference pattern, causing the spectrum to shift in wavelength and/or amplitude (depending on sensor construction); this signal returns to the circulator by a reflective facet 8 at the terminus. Alternatively, the reflecting facet can be applied to the SMF to terminate at any convenient location past the MCF sensor and in the SMF. Alternatively, the optical signal can return to the optical circulator without a reflector simply by providing an additional length of SMF that returns to the circulator.

The MCF sensor is more sensitive than conventional electrical (Wheatstone bridge, etc.) strain sensors, Michelson interferometer-based strain sensors, Mach-Zender interferometric techniques, or even Fabry Perot fiber optic strain sensors, therefore the MCF strain sensor is the preferred embodiment. As described in the patent issued for the MCF optic sensor, U.S. Pat. No. 9,810,557, MULTICORE OPTICAL FIBER APPARATUS, METHODS, and APPLICATIONS [3], which is the present invention benefits from, the MCF sensor is fabricated by splicing a segment of particularly characterized MCF between two segments (input, output) of a standard single mode, single core fiber (SMF). The excitation of various modes in the MCF by the single mode input fiber produces a periodic modulation of the spectral response of the device due to multimode interference in the MCF. The modulation may have up to a 50 dB dynamic range depending on the design of the MCF. Changes in certain physical parameters of interest of an external environment in which the device is being used will affect the multimode interference pattern, causing the spectrum to shift in wavelength. Monitoring this shift in the spectrum allows for accurate measurement of the physical parameters of interest, which include but are not limited to temperature, pressure, strain, vibration, and sound, in real time. In the case of the present invention, as described above, strain is introduced via mechanical or kinematic amplification assembly to physically translate this crystallographic distortion to a positive or negative strain event whereupon the MCF responds through this physical contact linkage into crystallographic distortions in real time; these distortions occur when the environment around the sensor has varying concentrations of hydrogen. Although the MCF optical sensor is among the most bend/strain sensitive sensor design known and is the preferred embodiment of the invention, virtually any strain sensor arrangement can be implemented for hydrogen detection by using mechanical or kinematic amplification methods as long as the hydride is present and mechanically or kinematically connected to provide inputs into the strain sensor system to so that, once properly calibrated, hydrogen concentration can be deduced. There may even be some environments where no mechanical or kinematic amplification method is required, and the strain sensor—irrespective of the type—can be applied directly on to the hydride material.

In the case of the inventive sensor preferred embodiment, the mechanically amplified MCF hydrogen sensor, referring to FIG. 4, a suitably fixtured reactive hydride material 9, constructed such that crystallographic changes from variations in hydrogen concentration translate along the linear axis, thereby actuating the mechanical amplification arm 10, which then impinges upon the MCF sensor 11. Because MCF sensor 11 is fixed at one end and in a state of constant contact with arm 10, the reversible and uneven induced strain relates back to hydrogen concentration around the sensor. This is not the only possible arrangement since the active MCF portion of the fiber can be at the tip or along the length of the optical fiber; the fiber can therefore be fixed on one end or both ends. The MCF signal can be reflected to the circulator in the case of a MCF sensor with a reflecting facet, or the fiber can be continuous and lead back to the circulator with no reflection needed. This induced curvature from the mechanical actuation will result in a change to the entire MCF spectrum, either in peak wavelength or intensity, depending on the MCF construction. Due to the high contrast shape and sinusoidal features of the spectrum a narrow wavelength bandwidth of the spectrum can yield a large intensity change to a simple photodiode.

The ability of the MCF sensor to respond to the bending force imparted into the optical fiber substrate through the mechanical amplification mechanism provides the basis for environmental detection. The MCF sensor typically consists of a 5- to 20-millimeter-long segment of seven-core fiber spliced between two single-mode fibers, although other lengths and multicore arrangements are possible. When the optical fiber device is kept straight and unstrained, a pronounced interference pattern appears in the transmission spectrum. However, when the sensor is bent, a change to the interference pattern is produced, and the visibility of the interference notches changes. This allows for using either intensity or spectral shift for sensor interrogation. The dynamic range of the device can be tailored through the proper selection of the length of the seven-core fiber, although any closely coupled multicore fiber arrangement can be used. Linear sensitivity of about 3000 nm/mm⁻¹ for bending is observed experimentally. Given the point-sensor end of the MCF is free, and the other end towards the light source is fixtured and constrained, the ability of the sensor to detect small movements delivered by the amplification mechanism are assured with fine tuning being a function of the desired measurement range.

The inventive sensor benefits from recently defined, highly optimized hydrogen absorbing materials. While existing materials such as Pd, LaNi₅, TiFe, Ti—V—Cr, and Mg₄NiPd exhibit good thermodynamic features for room temperature hydrogen storage, these materials either require a complex thermal activation process or they suffer from high price and poor air resistance. Using these hydrides requires an activation process that involves heat cycling application for subsequent hydrogen absorption (activation and kinetic problem) and/or hydrogen desorption (thermodynamic stability problem). The preferred materials, taken advantage of by the inventive sensor, on the other hand, include high-entropy alloys (HEAs), which consist of five or more elements in equal or close to equal atomic fractions [4]. TiZrCrMnFeNi is one such alloy, and its properties include a cell volume in the hydride phase being 40% larger than the cell volume of the metallic phase due to the presence of hydrogen atoms in the lattice. This is key to the inventive sensor's mechanism of operation that relies on physically straining the benefitting MCF sensor. Additionally, kinetic measurements confirm that the material absorbs hydrogen extremely fast, readily forming TiZrCrMnFeNiH₆ in less than one minute. Furthermore, these alloys do not require inert environments storage since they have good resistance to the atmosphere and can be handled and stored in the air which further adds to the utility of the invention.

1. An improved fiber-optic hydrogen point sensor, comprising a device assembly with at least three (3) components, including:
a) a hydridable substance such as hydride presented so that hydrogen uptake and release-related crystallographic distortion is readily measurable across at least one axis or dimension, and
b) an optional mechanical or kinematic amplification mechanism, and
c) a strain sensor with design options including optical fiber, specifically multicore fiber (MCF) or Fabry Perot fiber-optic interferometric strain sensors; or Mach-Zehnder interferometric sensors, or Michelson interferometric sensors, or conventional Wheatstone bridge electrical strain sensors, or even electromagnetic radiation-based displacement sensors that are able to measure strain indirectly, including but not limited to millimeter wave or laser-based displacement detection mechanisms, or 3D scanning technologies.

2. The MCF optical strain sensor of claim 1 further characterized by
a. a 5 to 20 mm length of MCF with a terminating reflecting facet, this facet found in the MCF comprising the terminus (SMF-MCF-reflecting facet), or in an additional length of single mode fiber (SMF) fusion spliced to the MCF which comprises the terminus (SMF-MCF-SMF-reflecting facet), optically connected to a light source and monitoring apparatus through a circulator, together capable of providing calibratable spectral signal information generated by the MCF's response to hydrogen concentration;
b. an MCF sensor in contact through a kinematic or mechanical amplification mechanism with what is otherwise traditionally classified as a pure metal or alloy that exhibits favorable hydrogen uptake and release characteristics, and therefore considered a hydride, or hydridable material;
c. an MCF sensor in contact through a kinematic or mechanical amplification mechanism with a hydridable material that exhibits a notable change in unit cell volume upon hydrogen uptake or release;
d. an MCF sensor in contact through a kinematic or mechanical amplification mechanism with a hydridable material that maximizes the translation of a change in volume of the hydridable material to the MCF, yielding an optimized curvature or bend to the MCF;
e. an MCF in contact through a kinematic or mechanical amplification mechanism with a material in the class of high-entropy alloys, defined as alloys containing five or more elements in equal or close to equal atomic fractions, and forming the C14 Laves phase when the hydride is generated;
f. an optional sensor housing fixture designed to optimize sample-sensor interaction including a housing defining an interior section with an inlet port and an exit port, the housing being configured to accommodate the flow of a gas stream from the inlet port to the exit port.

3. The improved fiber-optic hydrogen sensor of claim 1, wherein the length segment of the MCF having a central optical core and at least six optical cores disposed circumferentially about the central core, and a cladding having a refractive index, nc, disposed there around, wherein each of the cores is characterized by a refractive index, ni, each of which ni is greater than nc; and
a. one or optionally two respective length segments of the single mode fiber (SMF) having a single, central optical core and a cladding there around,
b. wherein the length segment of the MCF is disposed intermediate the one or two respective length segments of the SMF in a serial SMF-MCF-SMF relationship, wherein the central core of the length segment of the MCF is in optical alignment with the central cores of the two respective length segments of the SMFs,
c. wherein a reflecting facet can terminate the SMF-MCF or SMF-MCF-SMF optical sensor.

4. The kinematic or mechanical amplification mechanism of claim 1 consisting of a flexure-based displacement amplifier, including bridge-type, lever-type, or triangular-type

5. The fiber optic sensor of claim 2, wherein light propagated through the (SMF-MCF-SMF) set is characterized by a spectrally periodic transmission modulation due to multimode interference of the propagated light in the length segment of the MCF.

6. The fiber optic sensor of claim 2, wherein the periodic modulation has a dynamic range between 1 dB to 50 dB.

7. The fiber optic sensor of claim 2, wherein any of the central core and any of the at least six circumferentially disposed optical cores of the MCF have a separation distance between 0.5 micrometers (μm) and 6 μm.

8. The fiber optic sensor of claim 2, wherein the at least six optical cores have a symmetric distribution about the central optical core of the MCF.

9. The fiber optic sensor of claim 2, wherein each of the at least seven optical cores of the MCF are Ge-doped cores embedded in a solid silica cladding.

10. The fiber optic sensor of claim 2, wherein each of the at least seven optical cores of the MCF have a core diameter in the range from 4 μm to 16 μm.

11. The fiber optic sensor of claim 2, wherein each of the at least seven optical cores have a numerical aperture (NA) in the range from 0.05 to 0.18.

12. The fiber optic sensor of claim 2, wherein at least some of the ni values are the same.

13. A method of using a fiber optic sensor comprising:
a. providing a single mode optical fiber (SMF)-multiple core optical fiber (MCF)-single mode optical fiber (SMF-MCF-SMF) set having serially/optically-aligned central cores;
b. inputting a fundamental mode propagating in a leading one of the SMFs into the MCF; and
c. exciting between two to eight supermodes in the MCF propagating therein comprising a periodic modulation of a spectral response of the sensor due to multimode interference of the two to eight supermodes in the MCF.

14. The method of claim 13, further comprising detecting a change in the MCF spectral response which is a function of bend or curvature initiated by subtle dimensional changes of the active hydride material and optionally transmitted via a kinematic or mechanical amplification mechanism operationally disposed to relay and transmit these changes from the hydride material to the MCF.
